(19) United States
(12) Patent Application Publication
(10) Pub. No.: US 2002/0009799 A1
(43) Pub. Date: Jan. 24, 2002

(54) Isolation and purification of nucleic acids

(76) Inventors: Randal A. Goffe, Arlington, WA (US); Andrae D. Goffe, Arlington, WA (US); Ronald W. Berninger, Mukilteo, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 09/733,240

(22) Filed: Dec. 8, 2000

Related U.S. Application Data
(60) Provisional application No. 60/170,185, filed on Dec. 10, 1999.
(60) Provisional application No. 60/241,638, filed on Oct. 19, 2000.

(51) Int. Cl. C12N 1/08

(52) U.S. Cl. 435/320.1; 435/270

(57) ABSTRACT

In one aspect the present invention provides methods for isolating nucleic acid molecules from a cell, the methods comprising (a) contacting a cell with a solution comprising a biopolymer-degrading enzyme, provided that the biopolymer-degrading enzyme is not a nuclease, and (b) contacting the cell with a solution comprising a hydrophobic surfactant to yield a cell suspension comprising cell, biopolymer-degrading enzyme and hydrophobic surfactant, wherein the hydrophobic surfactant has a critical micelle concentration less than 3.0 mM and the concentration of the hydrophobic surfactant in the cell suspension is at least 0.05% (v/v). In another aspect the present invention provides isolated DNA preparations comprising at least 80% supercoiled DNA. In another aspect the present invention provides isolated nucleic acid preparations having an A_{260/230} ratio of at least 2.0.

### RELATED APPLICATIONS

This application claims benefit of priority from U.S. Provisional Patent Application No. 60/170,185, filed Dec. 10, 1999, and from U.S. Provisional Patent Application No. 60/241,638, filed Oct. 19, 2000, benefit of priority from which applications is claimed under 35 U.S.C. §119.

### FIELD OF THE INVENTION

This invention relates to methods for isolating and purifying nucleic acids, such as DNA, and to isolated nucleic acids.

### BACKGROUND

Isolation of nucleic acid molecules, such as DNA and RNA, from cells typically involves suspending the cells in a suspension buffer to which is added a lysis buffer that breaks the cells open, i.e., lyses the cells.. A typical lysis solution is alkaline and contains a non-hydrophobic surfactant, such as sodium dodecyl sulfate (SDS). See, e.g., Molecular Cloning: A Laboratory Manual, J. Sambrook, E. F. Fritsch, T. Maniatis, eds, 2^{nd} edition, ps. 1.25-1.28, Cold Spring Harbor Laboratory (1989). After the cell contents have been released, unwanted cellular debris, such as proteins, are at least partially removed, for example by centrifugation or filtration.

Contamination of nucleic acids with other cellular components upon lysis remains a problem, however, and can impair the nucleic acid yield, and the functional properties of the isolated nucleic acids. For example, the inventors have observed that contamination of plasmid DNA isolated from bacteria can reduce the effectiveness of the plasmid DNA as a template for transient transcriptional expression in vivo and in vitro. Possible contaminants include lipopolysaccharides, phospholipids, glycerophospholipids, polysaccharides, proteoglycans and proteins.

Moreover, the release of large biopolymer molecules during cell lysis increases the viscosity of the lysate, thereby requiring the application of strong shear forces to mix the lysate during subsequent extraction of the nucleic acids. These strong shear forces tend to break the nucleic acid chains, further reducing their usefulness as templates for transcription or other enzymatic reactions. Further, degraded nucleic acid molecules become contaminants in the lysate, and products derived therefrom.

There is therefore a need for methods for isolating nucleic acid molecules from cells that yield intact nucleic acid molecules that are free, or substantially free, from contaminants.

### SUMMARY OF THE INVENTION

In one aspect the present invention provides methods for isolating nucleic acid molecules from a cell, the methods comprising (a) contacting a cell with a solution comprising a biopolymer-degrading enzyme, provided that the biopolymer-degrading enzyme is not a nuclease; and (b) contacting the cell with a solution comprising a hydrophobic surfactant to yield a cell suspension comprising cell, biopolymer-degrading enzyme and hydrophobic surfactant, wherein the hydrophobic surfactant has a critical micelle concentration less than 3.0 mM (e.g., less than 2 mM, or less than 1 mM) and the concentration of the hydrophobic surfactant in the cell suspension is at least 0.05% (v/v) (e.g., at least 0.10% (v/v), at least 0.15% (v/v), or at least 0.20% (v/v)). In some embodiments the solution comprising a hydrophobic surfactant further comprises a non-hydrophobic surfactant, wherein the non-hydrophobic surfactant has a critical micelle concentration greater than 3.0 mM, and the concentration of the non-hydrophobic surfactant in the cell suspension is at least 0.4% (v/v).

In another aspect, the present invention provides methods for isolating nucleic acid molecules from a cell comprising: (a) suspending a cell in a solution comprising a carbohydrate-degrading enzyme to form a cell suspension; (b) adding to the cell suspension (1) an amount of at least one hydrophobic surfactant sufficient to yield a hydrophobic surfactant concentration of at least 0.05% (v/v), the hydrophobic surfactant having a critical micelle concentration of less than 3.0 mM, and (2) an amount of an alkaline agent sufficient to increase the pH of the cell suspension to a pH value greater than 10.0; and (c) adding to the cell suspension prepared in accordance with steps (a) and (b) an amount of a neutralizing agent sufficient to adjust the pH of the cell suspension to within the range of from pH 6.5 to pH 7.5. In some embodiments, step (b) further comprises adding to the cell suspension of step (a) an amount of a non-hydrophobic surfactant sufficient to yield a non-hydrophobic surfactant concentration of at least 0.4% (v/v), wherein the non-hydrophobic surfactant has a critical micelle concentration greater than 3.0 mM.

In another aspect, the present invention provides isolated nucleic acid preparations, prepared in accordance with the methods of the invention, having an A_{260/230} ratio of at least 2.0. In yet another aspect, the present invention provides isolated DNA preparations comprising at least 80% supercoiled DNA (such as at least 90% or at least 95% supercoiled DNA).

In other aspects, the present invention provides isolated plasmid DNA having desirable expression characteristics as described more fully herein.

The methods of the invention are useful for isolating nucleic acid molecules in any situation where isolated nucleic acid molecules are desired. Similarly, the isolated nucleic acid molecules (such as isolated plasmid DNA) of the invention are useful in any situation where isolated nucleic acid molecules are desired. Thus, for example, the methods and compositions of the invention are useful for providing isolated DNA that can be introduced into prokaryotic or eukaryotic cells in vivo or in vitro to inhibit, enhance, or otherwise modify gene expression within the cells. For example, the methods of the invention can be used to isolate plasmid DNA that is introduced into mammalian cells in vivo wherein one or more proteins encoded by the plasmid DNA are expressed and confer a desirable phenotype on the cells. Exemplary uses of the isolated nucleic acid molecules of the invention are: to construct DNA vectors, to transform, transfect or otherwise genetically modify living cells in vivo or in vitro, and to function as nucleic acid probes for identifying cDNAs or genes of interest.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows plots of optical density (measured at a wavelength of 600 nanometers) of lysis solutions of the invention that each include the same amount of a hydrophobic surfactant (1% OT-100) and different amounts of a non-hydrophobic surfactant (SDS), in the presence (diamond symbols) or absence (square symbols) of equal amounts of an Escherichia coli cell lysate. A cell suspension buffer including 1% (v/v) sodium dodecyl sulfate was used to set the zero value of the spectrophotometer.

FIG. 2 shows a representative scheme for isolating nucleic acid molecules from E. coli utilizing one embodiment of the methods of the invention. The abbreviation “SLC” means synthetic ligand compounds.

FIG. 3 shows a plot of viscosity in units of milliPascals (mPa) versus shear rate (1/s) for a suspension solution composed of 50 mM dextrose, 26 mM Tris, 10 mM EDTA, pH 8.0. Diamond symbols represent viscosity values obtained by increasing the shear rate. Square symbols represent viscosity values obtained by decreasing the shear rate.

FIG. 4 shows a plot of viscosity (mPa) versus shear rate (1/s) for the suspension solution described in the legend to FIG. 3, and further comprising 50 units per milliliter β-amylase. Diamond symbols represent viscosity values obtained by increasing the shear rate. Square symbols represent viscosity values obtained by decreasing the shear rate.

FIG. 5 shows a plot of viscosity (mPa) versus shear rate (1/s) for a neutralization solution composed of 3.1 M potassium acetate, pH 5.5. Diamond symbols represent viscosity values obtained by increasing the shear rate. Square symbols represent viscosity values obtained by decreasing the shear rate.

FIG. 6 shows a plot of viscosity (mPa) versus shear rate (1/s) for a lysis solution composed of 0.4% SDS, 0.2% S 465, 0.2 N NaOH. Diamond symbols represent viscosity values obtained by increasing the shear rate. Square symbols represent viscosity values obtained by decreasing the shear rate.

FIG. 7 shows a plot of viscosity (mPa) versus shear rate (1/s) for a lysis solution composed of 0.4% SDS, 0.2 N NaOH, 0.2% OT-100. Diamond symbols represent viscosity values obtained by increasing the shear rate. Square symbols represent viscosity values obtained by decreasing the shear rate.

FIG. 8 shows a plot of viscosity (mPa) versus shear rate (1/s) for a lysis solution composed of 1.0% SDS, 0.2 N NaOH. Diamond symbols represent viscosity values obtained by increasing the shear rate. Square symbols represent viscosity values obtained by decreasing the shear rate.

FIG. 9 shows a plot of viscosity (mPa) versus shear rate (1/s) for a lysis solution composed of 0.4% SDS, 0.2 N NaOH. Diamond symbols represent viscosity values obtained by increasing the shear rate. Square symbols represent viscosity values obtained by decreasing the shear rate.

FIG. 10 shows a plot of viscosity (mPa) versus shear rate (1/s) for a lysis solution composed of 0.4% SDS, 0.2 N NaOH, 0.2% S-485. Diamond symbols represent viscosity values obtained by increasing the shear rate. Square symbols represent viscosity values obtained by decreasing the shear rate.

FIG. 11 shows a plot of viscosity (mPa) versus shear rate (1/s) for a mixture of a lysis solution composed of 1.0% SDS, 0.2 N NaOH; a suspension solution composed of 50 mM dextrose, 26 mM Tris, 10 mM EDTA, pH 8.0, 50 units per milliliter β-amylase; and an E. coli cell lysate. Diamond symbols represent viscosity values obtained by increasing the shear rate. Square symbols represent viscosity values obtained by decreasing the shear rate.

FIG. 12 shows a plot of viscosity (mPa) versus shear rate (1/s) for a mixture of a lysis solution composed of 0.4% SDS, 0.2 N NaOH; a suspension solution composed of 50 mM dextrose, 26 mM Tris, 10 mM EDTA, pH 8.0, 50 units per milliliter β-amylase; and an E. coli cell lysate. Diamond symbols represent viscosity values obtained by increasing the shear rate. Square symbols represent viscosity values obtained by decreasing the shear rate.

FIG. 13 shows a plot of viscosity (mPa) versus shear rate (1/s) for a mixture of a lysis solution composed of 0.4% SDS, 0.2 N NaOH, 0.2% S-485; a suspension solution composed of 50 mM dextrose, 26 mM Tris, 10 mM EDTA, pH 8.0, 50 units per milliliter β-amylase; and an E. coli cell lysate. Diamond symbols represent viscosity values obtained by increasing the shear rate. Square symbols represent viscosity values obtained by decreasing the shear rate.

FIG. 14 shows a plot of viscosity (mPa) versus shear rate (1/s) for a mixture of a lysis solution composed of 0.4% SDS, 0.2 N NaOH, 0.2% S-465; a suspension solution composed of 50 mM dextrose, 26 mM Tris, 10 mM EDTA, pH 8.0, 50 units per milliliter β-amylase; and an E. coli cell lysate. Diamond symbols represent viscosity values obtained by increasing the shear rate. Square symbols represent viscosity values obtained by decreasing the shear rate.

FIG. 15 shows a plot of viscosity (mPa) versus shear rate (1/s) for a mixture of a lysis solution composed of 0.4% SDS, 0.2 N NaOH, 0.2% OT-100; a suspension solution composed of 50 mM dextrose, 26 mM Tris, 10 mM EDTA, pH 8.0, 50 units per milliliter β-amylase; and an E. coli cell lysate. Diamond symbols represent viscosity values obtained by increasing the shear rate. Square symbols represent viscosity values obtained by decreasing the shear rate.

FIG. 16 shows a plot of viscosity (mPa) versus shear rate (1/s) for the mixture of lysis and suspension solutions, described in the legend to FIG. 11, that have been neutralized with 3.1 M potassium acetate, pH 5.5. Diamond symbols represent viscosity values obtained by increasing the shear rate. Square symbols represent viscosity values obtained by decreasing the shear rate.

FIG. 17 shows a plot of viscosity (mPa) versus shear rate (1/s) for the mixture of lysis and suspension solutions, described in the legend to FIG. 12, that have been neutralized with 3.1 M potassium acetate, pH 5.5. Diamond symbols represent viscosity values obtained by increasing the shear rate. Square symbols represent viscosity values obtained by decreasing the shear rate.

FIG. 18 shows a plot of viscosity (mPa) versus shear rate (1/s) for the mixture of lysis and suspension solutions, described in the legend to FIG. 13, that have been neutralized with 3.1 M potassium acetate, pH 5.5. Diamond symbols represent viscosity values obtained by increasing the shear rate. Square symbols represent viscosity values obtained by decreasing the shear rate.

FIG. 19 shows a plot of viscosity (mPa) versus shear rate (1/s) for the mixture of lysis and suspension solutions, described in the legend to FIG. 14, that have been neutralized with 3.1 M potassium acetate, pH 5.5. Diamond symbols represent viscosity values obtained by increasing the shear rate. Square symbols represent viscosity values obtained by decreasing the shear rate.

### DETAILED DESCRIPTION OF THE PREFERRED EMBODIMENT

In accordance with the foregoing, in one aspect the present invention provides methods for isolating nucleic acid molecules from a cell, the methods comprising (a) contacting a cell with a solution comprising a biopolymer-degrading enzyme, provided that the biopolymer-degrading enzyme is not a nuclease; and (b) contacting the cell with a solution comprising a hydrophobic surfactant to yield a cell suspension comprising cell, biopolymer-degrading enzyme and hydrophobic surfactant, wherein the hydrophobic surfactant has a critical micelle concentration less than 3.0 mM (e.g., less than 2 mM, or less than 1 mM) and the concentration of the hydrophobic surfactant in the cell suspension is at least 0.05% (v/v) (e.g., at least 0.10% (v/v), at least 0.15% (v/v), or at least 0.20% (v/v)).

As used herein, the term “critical micelle concentration”, abbreviated as CMC, is the concentration of a molecule, such as a hydrophobic surfactant or non-hydrophobic surfactant, above which micelles are formed. See, e.g., Journal of Biological Chemistry 251: 4442 (1976); Biochim Biophys Acta 455: 796 (1976); Biochim Biophys Acta 553: 40 (1979), incorporated herein by reference.

As used herein, the term “nuclease” means a molecule that breaks down nucleic acid molecules (such as DNAse proteins that break down DNA molecules, and RNAse proteins that break down RNA molecules).

As used herein, the term “biopolymer-degrading enzyme” refers to enzymes that degrade polymers that are synthesized by living cells, such as proteins, carbohydrates and lipids. Any enzyme that degrades one or more types of biopolymers is useful in this aspect of the invention. Representative examples of carbohydrate-degrading enzymes useful in this aspect of the invention (or in any aspect of the invention that utilizes a carbohydrate-degrading enzyme) include: α-amylase, β-amylase, amyloglucosidase, invertase and glycopepsidase F. While not wishing to be bound by theory, it is believed that degradation of biopolymers, especially those associated with the exterior surface of the cell, early in the nucleic acid extraction process reduces the viscosity of the resulting solution. This reduction in viscosity allows relatively gentle mixing to be used during nucleic acid extraction, thereby minimizing shearing of nucleic acid molecules. Additionally, some major contaminants (e.g., mucopolysaccharides of bacterial cell wall origin) can be substantially removed early in the extraction process.

In some embodiments of this aspect of the invention, the solution comprising a biopolymer-degrading enzyme and the solution comprising a hydrophobic surfactant are the same solution. In other embodiments of this aspect of the invention, the solution comprising a biopolymer-degrading enzyme and the solution comprising a hydrophobic surfactant are different solutions. In some embodiments in which the solution comprising a biopolymer-degrading enzyme and the solution comprising a hydrophobic surfactant are different solutions, the cell is first contacted with the solution comprising a biopolymer-degrading enzyme, and then the cell is next contacted with the solution comprising the hydrophobic surfactant. In other embodiments in which the solution comprising a biopolymer-degrading enzyme and the solution comprising a hydrophobic surfactant are different solutions, the cell is contacted with the solution comprising a biopolymer-degrading enzyme at the same time that the cell is contacted with the solution comprising the hydrophobic surfactant.

Typically the concentration of hydrophobic surfactant in the cell suspension is no greater than 1% (v/v). Typically, the cell suspension has an alkaline pH, typically within the range of from pH 10.0 to pH 11.0. The hydrophobic surfactant promotes lysis of the one or more cells, and binds hydrophobic molecules. It is understood that typically a plurality of cells are treated in accordance with the methods of the invention.

The hydrophobic surfactants useful in the practice of the present invention are more soluble in non-aqueous solvents than in aqueous solvents, and can bear an overall positive charge, an overall negative charge, or be uncharged. The hydrophobic surfactants useful in the practice of the present invention tend to bind strongly to hydrophobic molecules and surfaces.

The hydrophobic surfactants useful in the practice of the present invention have a critical micelle concentration less than 3.0 mM. Exemplary values for the critical micelle concentration of hydrophobic surfactants useful in the practice of the present invention are less than 2.0 mM, less than 1.0 mM, less than 0.5 mM, and less than 0.1 mM.

Some hydrophobic surfactants useful in the practice of the present invention have a hydrophile lipophile balance number of less than 20. Other hydrophobic surfactants useful in the practice of the present invention have a hydrophile lipophile balance number of less than 15, or less than 10. As used herein, the term “hydrophilic-lipophilic balance number”, abbreviated as HLB number, is an indicator of the hydrophilic character of a molecule, such as a hydrophobic surfactant or non-hydrophobic surfactant; the larger the HLB, the more hydrophilic the molecule. See, e.g., Journal of Biological Chemistry 251: 4442 (1976); Biochim Biophys Acta 455: 796 (1976); Biochim Biophys Acta 553: 40 (1979).

Some hydrophobic surfactants useful in the practice of the present invention have a solubility in water of less than 2 grams/100 milliliters (2 g/100 mL) Exemplary values for the solubility in water of hydrophobic surfactants useful in the practice of the present invention are less than 1.5 g/100 mL and less than 1.0 g/100 mL.

Table 1 sets forth some properties of representative hydrophobic surfactants useful in the practice of the present invention. Note that sodium dodecyl sulfate (SDS) and CHAPS are non-hydrophobic surfactants included for comparison. An additional, representative, anionic, hydrophobic surfactant is TR-70 (sodium bistridecyl sulfosuccinate) which has an equilibrium surface tension of 27 dynes/cm, a CMC of 0.0005 mM to 0.0015 mM, and a solubility in water of 0.15%.

TABLE I
CRITICAL SURFACTANT PROPERTIES AND BIOLOGICAL APPLICATIONS

| | | | | | | | | | Dynamic |
|---|---|---|---|---|---|---|---|---|---|
| | | | | | | | | | Surface |
| | | | | | | Cloud | | Equilibrium | Tension |
| | | | CMC | Aggregation | | Point | Solubility (in | Surface Tension | (dynes/cm)* |
| | Composition | | (mM)^{(A)} | Number^{(B)} | HLB^{(C)} | (° C.)^{(D)} | water?) | (dynes/cm) | [6 bubbles/sec] |
| ANI | | | | | | | | | |
| ONI | | | | | | | | | |
| Aerosol ® OT | Sodium dioctyl sulfosuccinate | | 0.1 | — | 15 | >100 | 1.5 g/100 mL | 30.8⁺ | 32.8⁺ |
| SDS | Sodium dodecyl sulfate, | | 7-10 | 62 | 40 | >100 | — | — | — |
| NONIONIC | | | | | | | | | |
| Tween ® 20 | Polyoxyethylene sorbitan | | 0.06 | — | 16.7 | 76 | — | — | — |
| | monolaurate | | | | | | | | |
| Tween ® 80 | Polyoxyethylene sorbitan | | 0.012 | 60 | 15 | 65 | — | — | — |
| | monooleate | | | | | | | | |
| Brij ® 35 | Polyoxyethylene 23 | | 0.05-0.1 | 20-40 | 16.9 | >100 | — | — | — |
| | lauryl ether | | | | | | | | |
| Surfynol ® 420 | Weight % | 20 | N/A | — | 4 | N/A | 0.1-1.0% | 32.0 | — (35.1) |
| Surfynol ® 440 | ethylene | 40 | N/A | — | 6-7 | N/A | 0.1-1.0% | 33.2 | 35.0 (37.3) |
| Surfynol ® 465 | glycol adduct | 65 | 0.65 | — | 13 | 63 | >1.0% | 38.0 | 40.2 (44.3) |
| Surfynol ® 485 | to acetylenic | 85 | 1.65 | — | 17 | >100 | >1.0% | 51.1 | — (53.0) |
| | diol | | | | | | | | |

| | | | | | | | | |
|---|---|---|---|---|---|---|---|---|
| Triton ® X-100 | | 0.2-0.5 | 100-155 | 13.5 | 65 | — | — | — (42.2) |
| Triton ® X-114 | | 0.2 | — | 12.4 | 23 | — | — | — |
| ZWITTER- | | | | | | | | |
| IONIC | | | | | | | | |
| CHAPS | 3-[(3-Cholamidopropyl)- | 6 | 10 | — | >100 | — | — | — |
| | dimethylammonio]-1- | | | | | | | |
| | propanesulfonate) | | | | | | | |
| SB-14 | (N-Tetradecyl-N,N- | 0.1-0.4 | 83 | — | — | — | — | — |
| | dimethyl-3-ammonio-I- | | | | | | | |
| | propanesulfonate 3) | | | | | | | |

* Surfactants that diffuse or migrate rapidly in aqueous media have low dynamic surface tension. [See Joel Schwartz, “The Importance of Low Dynamic Surface Tension in Waterborne Coating”, J. Coatings Technology, 1992, 64, (812) p. 65-74]. ⁺ Based upon Aerosol® OT 75; 75% solid in water/EtOH. (A) Critical Micelle Concentration (CMC). (B) Aggregation Number: The number of monomers in a micelle. (C) Hydrophile-Lipophile Balance (HLB) Number. (D) Cloud Point: the temperature above which turbidity or phase separation occurs.

Some embodiments of the methods of the invention comprise the steps of: (a) contacting a cell with a solution comprising a biopolymer-degrading enzyme, provided that the biopolymer-degrading enzyme is not a nuclease; and (b) contacting the cell with a solution comprising a hydrophobic surfactant and a non-hydrophobic surfactant to yield a cell suspension comprising cell, biopolymer-degrading enzyme, hydrophobic surfactant and non-hydrophobic surfactant. The hydrophobic surfactant has a critical micelle concentration less than 3.0 mM (e.g., less than 2 mM, or less than 1 mM) and the concentration of the hydrophobic surfactant in the cell suspension is at least 0.05% (v/v) (e.g., at least 0.10% (v/v), at least 0.15% (v/v), or at least 0.20% (v/v)). The non-hydrophobic surfactant has a critical micelle concentration greater than 3.0 mM (e.g., greater than 5.0 mM, or greater than 7.0 mM) and the concentration of the non-hydrophobic surfactant in the cell suspension is at least 0.4% (v/v) (e.g., at least 0.5% (v/v), at least 0.6% (v/v), or at least 0.7% (v/v)). Typically the concentration of hydrophobic surfactant in the cell suspension is no greater than 1% (v/v), and the concentration of non-hydrophobic surfactant in the cell suspension is no greater than 2% (v/v).

The non-hydrophobic surfactants useful in the practice of the present invention are more soluble in aqueous solvents than in non-aqueous solvents, and can bear an overall positive charge, an overall negative charge, or be uncharged. While not wishing to be bound by theory, it is believed that the non-hydrophobic surfactants are more effective at lysing cells than the hydrophobic surfactants.

In some embodiments of the methods of the invention which utilize a non-hydrophobic surfactant, the non-hydrophobic surfactant has a hydrophile lipophile balance number of greater than 20 (such as greater than 30). In some embodiments of the methods of the invention which utilize a non-hydrophobic surfactant, the non-hydrophobic surfactant has a solubility in water of greater than 2 g/100 mL (such as greater than 4 g/100 mL or greater than 6 g/100 mL). Representative examples of non-hydrophobic surfactants, having a critical micelle concentration greater than 3.0, include sodium dodecyl sulfate (SDS), CHAPS and N-octyl-β-D-thioglucoside.

In another aspect, the present invention provides methods for isolating nucleic acid molecules from a cell comprising the steps of (a) suspending one or more cells in a solution comprising a carbohydrate-degrading enzyme to form a cell suspension; (b) adding to the cell suspension (i) an amount of at least one hydrophobic surfactant sufficient to yield a hydrophobic surfactant concentration of at least 0.05% (v/v) (such as at least 0.10% (v/v), at least 0.15% (v/v), or at least 0.20% (v/v)), the hydrophobic surfactant having a critical micelle concentration of less than 3.0 mM (such as less than 2.0 mM, less than 1.0 mM, less than 0.5 mM, or less than 0.1 mM), and (ii) an amount of an alkaline chemical (such as sodium hydroxide) sufficient to adjust the pH of the solution to a pH value of greater than 10.0; and (c) adding to the solution prepared in accordance with steps (a) and (b) an amount of a neutralizing agent sufficient to adjust the pH of the solution to within the range of from 6.5 to 7.5 pH units.

Typically, the cell is contacted with the hydrophobic surfactant for a period of from 3 minutes to 12 minutes (such as from 4 to 6 minutes) before adding the neutralizing agent. In one embodiment, the at least one hydrophobic surfactant and alkaline agent are combined in a single solution which is added to the suspended cells. The one or more carbohydrate-degrading enzymes, can be any enzyme that degrades one or more types of carbohydrate molecules, such as the carbohydrate-degrading enzymes listed supra. Typically, the concentration of the hydrophobic surfactant in the solution does not exceed 1%(v/v). In some embodiments of this aspect of the invention, the hydrophobic surfactant has a hydrophile lipophile balance number of less than 20 (such as less than 15). In some embodiments of this aspect of the invention, the hydrophobic surfactant has a solubility in water of less than 2 g/100 mL. Exemplary values for the solubility in water of hydrophobic surfactants useful in this aspect of the invention are less than 1.5 g/100 mL or less than 1.0 g/100 mL.

In the methods of this aspect of the invention, a non-hydrophobic surfactant can be optionally added to the suspended cells, wherein the non-hydrophobic surfactant has a critical micelle concentration greater than 3.0. The concentration of the non-hydrophobic surfactant in the cell suspension is at least 0.4% (v/v), but typically not greater than 2% (v/v).

Neutralizing agents useful in the practice of those aspects of the invention that utilize an alkaline lysis solution include acidic salts, such as sodium acetate, pH 5.5 or potassium acetate, pH 5.5, or ammonium acetate, pH 5.5.

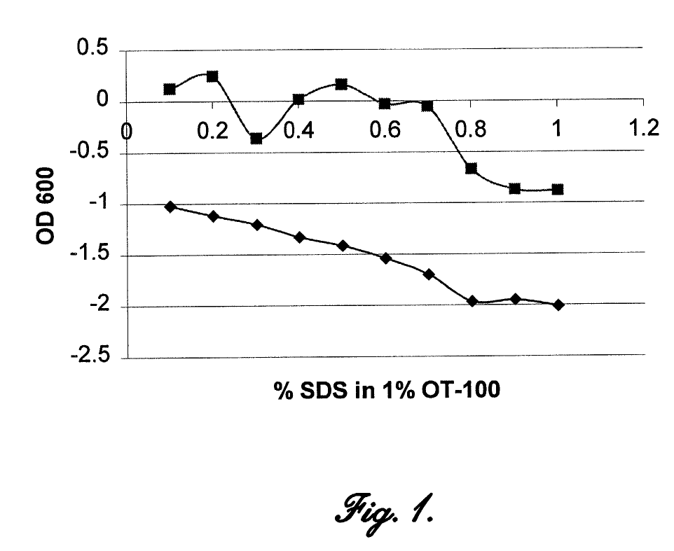

It is a feature of those embodiments of the methods of the present invention in which the one or more cells are contacted with a solution comprising (a) at least 0.05% (v/v) hydrophobic surfactant, wherein the hydrophobic surfactant has a critical micelle concentration less than 3.0; and (b) at least 0.4% (v/v) non-hydrophobic surfactant, wherein the non-hydrophobic surfactant has a critical micelle concentration greater than 3.0, that a phase-separated mixture is created in the presence of a cell lysate. For example, FIG. 1 shows plots of optical density (measured at a wavelength of 600 nanometers) of solutions of the invention that each include the same amount of a hydrophobic surfactant (1% OT-100) and different amounts of a non-hydrophobic surfactant (SDS), in the presence (diamond symbols) or absence (square symbols) of equal amounts of an Escherichia coli cell lysate. A marked phase separation occurs when the cell lysate is added to the solution, as shown by the decrease in OD₆₀₀, compared to the equivalent solution that does not include cell lysate.

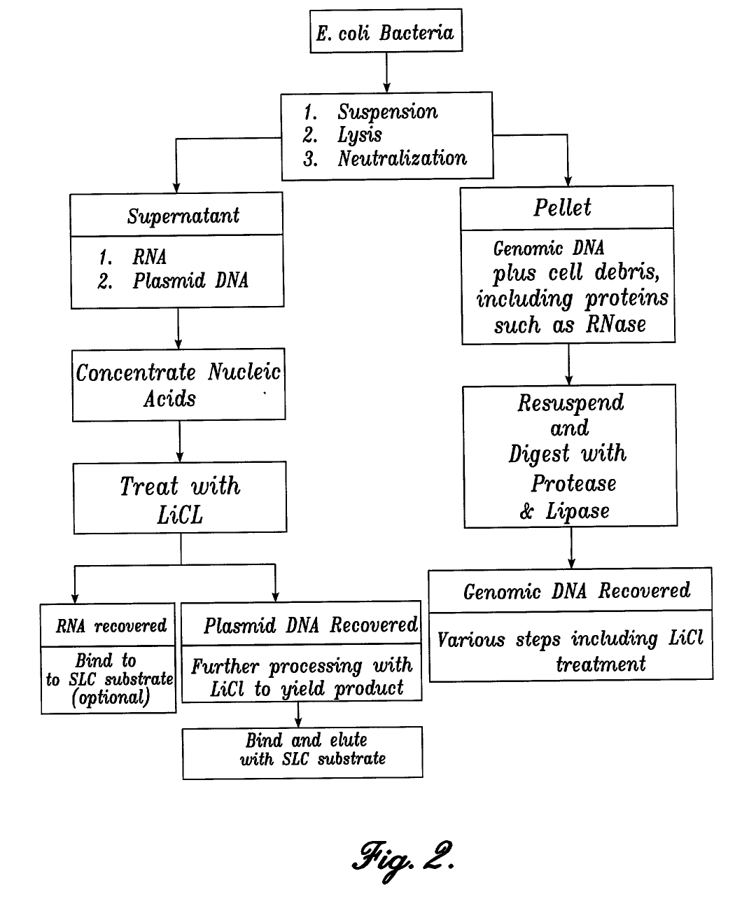

The methods of the invention are useful for isolating any type of nucleic acid molecule from any type of cell. Representative examples of cells that can be treated with the methods of the invention include prokaryotic (such as bacterial) and eukaryotic (such as mammalian and plant) cells. Representative examples of nucleic acid molecules that can be isolated from cells in accordance with the present invention include DNA (including genomic DNA and plasmid DNA) and RNA (including messenger RNA). FIG. 2 shows, by way of non-limiting example, a representative scheme for isolating nucleic acid molecules from E. coli utilizing one embodiment of the methods of the invention. The scheme shown in FIG. 2 includes: (1) the step of suspending the E. coli cells in a suspension solution that includes a carbohydrate-degrading enzyme; (2) the step of lysing the cells in an alkaline lysis solution that includes at least 0.05% (v/v) hydrophobic surfactant, wherein the hydrophobic surfactant has a critical micelle concentration less than 3.0; and (3) neutralizing the alkaline solution utilized in step (b) with a neutralization solution.

The methods of the present invention are capable of yielding highly pure preparations of DNA molecules as measured by the ratio of absorbance at 260 nanometers (A₂₆₀) to absorbance at 230 nanometers (A₂₃₀). Thus, in another aspect, the present invention provides isolated DNA preparations having an A_{260/230} ratio of at least 2.0 (such as isolated DNA preparations having an A_{260/230} ratio of at least 2.1, or at least 2.2). A₂₆₀ and A₂₃₀ are measured by dissolving the dried, isolated, DNA preparation in water and using a spectrophotometer, zeroed against water, to measure A₂₆₀ and A₂₃₀ of the dissolved DNA preparation. In the inventors' experience, the highest purity DNA preparations are achieved by those embodiments of the methods of the invention that include the steps of: (a) suspending one or more cells in a solution comprising a carbohydrate-degrading enzyme; (b) adding to the suspended cells an amount of at least one hydrophobic surfactant sufficient to yield a hydrophobic surfactant concentration of at least 0.05% (v/v), the hydrophobic surfactant having a critical micelle concentration of less than 3.0, and an amount of an alkaline agent (such as sodium hydroxide) to adjust the pH of the solution to an alkaline pH (typically in the range of from pH 10.0 to pH 11.0); and (c) adding to the cell suspension prepared in accordance with steps (a) and (b) an amount of a neutralizing agent sufficient to adjust the pH of the suspension to within the range of from 6.5 to 7.5 pH units.

Additionally, it has been found that plasmid DNA isolated in accordance with the present invention contains relatively few breaks in the phosphodiester backbone, i.e., contains few “nicks”, and so includes a low percentage of nicked, open-circular, plasmid DNA. Plasmid DNA that contains few nicks exists primarily as covalently closed circular, also known as supercoiled, DNA that migrates faster, during gel electrophoresis, through an agarose gel than does more highly nicked or linear plasmid DNA. Thus, in another aspect, the present invention provides isolated plasmid DNA comprising at least 80% supercoiled plasmid DNA. Some embodiments of this aspect of the invention provide isolated plasmid DNA comprising at least 90% supercoiled plasmid DNA, or isolated plasmid DNA comprising at least 95% supercoiled plasmid DNA. One way to measure the percentage of supercoiled plasmid DNA is to run a sample of plasmid DNA on an agarose gel using the technique of agarose gel electrophoresis. See, e.g., Molecular Cloning: A Laboratory Manual, J. Sambrook, E. F. Fritsch, T. Maniatis, eds, 2^{nd} edition, Chapter 6, Cold Spring Harbor Laboratory (1989). The gel is stained with ethidium bromide (which fluoresces under ultraviolet light), and the fluorescent intensity of the supercoiled DNA band (which migrates ahead of the nicked, open circular DNA band) is compared to the fluorescent intensity of the nicked, open circular DNA band.

In yet another aspect, the present invention provides isolated plasmid DNA that encodes a protein, and that, when introduced into a mammalian cell in vitro and expressed therein (such as in accordance with the method set forth in Example 5 herein), expresses the protein for a period of at least ten days (such as for 10 days, 15 days, 20 days, 25 days, 30 days, 35 days or 40 days) after introduction into the cell, the level of protein expression during the expression period never dropping below 50% of the peak protein expression level reached during the expression period.

In a related aspect, the present invention provides isolated plasmid DNA which is isolated from a cell by a method comprising contacting a cell with a solution comprising a biopolymer-degrading enzyme, provided that the biopolymer-degrading enzyme is not a nuclease, and contacting the cell with a solution comprising a hydrophobic surfactant to yield a cell suspension comprising cell, biopolymer-degrading enzyme and hydrophobic surfactant, wherein the hydrophobic surfactant has a critical micelle concentration less than 3.0 mM and the concentration of the hydrophobic surfactant in the cell suspension is at least 0.05% (v/v). The plasmid DNA so isolated possesses the property of expressing the protein for a period of time, after introduction into a mammalian cell in vivo, during which time period the level of protein expression (a) reaches a peak protein expression level and (b) never drops below 50% of the peak protein expression level. The time period of expression is at least two times longer than any period of expression of reference plasmid DNA, in the same type of mammalian cell in vivo, during which protein expression does not fall below 50% of the value of the peak protein expression level, the reference plasmid DNA being the same plasmid DNA as the plasmid DNA of the present invention except that the reference plasmid DNA is prepared by purification twice on a cesium chloride gradient instead of in accordance with the methods of the present invention.

Any art-recognized gene delivery method can be used to introduce the isolated plasmid DNA into one or more cells for expression therein, including: direct injection, electroporation, virus-mediated gene delivery, amino acid-mediated gene delivery, biolistic gene delivery, lipofection and heat shock. Non-viral methods of gene delivery into cells are disclosed in Huang, L., Hung, M-C, and Wagner, E., Non-Viral Vectors for Gene Therapy, Academic Press, San Diego, Calif. (1999), which is incorporated herein by reference.

For example, genes can be introduced into cells in situ, or after removal of the cells from the body, by means of viral vectors. For example, retroviruses are RNA viruses that have the ability to insert their genes into host cell chromosomes after infection. Retroviral vectors have been developed that lack the genes encoding viral proteins, but retain the ability to infect cells and insert their genes into the chromosomes of the target cell (A. D. Miller, Hum. Gen. Ther. 1:5-14 (1990)).

Adenoviral vectors are designed to be administered directly to patients. Unlike retroviral vectors, adenoviral vectors do not integrate into the chromosome of the host cell. Instead, genes introduced into cells using adenoviral vectors are maintained in the nucleus as an extrachromosomal element (episome) that persists for a limited time period. Adenoviral vectors will infect dividing and non-dividing cells in many different tissues in vivo including airway epithelial cells, endothelial cells, hepatocytes and various tumors (B. C. Trapnell, Adv Drug Del Rev. 12:185-199 (1993)).

Another viral vector is the herpes simplex virus, a large, double-stranded DNA virus that has been used in some initial applications to deliver therapeutic genes to neurons and could potentially be used to deliver therapeutic genes to some forms of brain cancer (D. S. Latchman, Mol. Biotechnol. 2:179-95 (1994)). Recombinant forms of the vaccinia virus can accommodate large inserts and are generated by homologous recombination. To date, this vector has been used to deliver interleukins (ILs), such as human IL-1β and the costimulatory molecules B7-1 and B7-2 (G. R. Peplinski et al., Ann. Surg. Oncol. 2:151-9 (1995); J. W. Hodge et al., Cancer Res. 54:5552-55 (1994)).

Another approach to gene therapy involves the direct introduction of DNA plasmids into patients. (F. D. Ledley, Hum. Gene Ther. 6:1129-1144 (1995)). The plasmid DNA is taken up by cells within the body and can direct expression of recombinant proteins. Typically plasmid DNA is delivered to cells in the form of liposomes in which the DNA is associated with one or more lipids, such as DOTMA (1,2,-diolcyloxypropyl-3-trimethyl ammonium bromide) and DOPE (dioleoylphosphatidylethanolamine). Formulations with DOTMA have been shown to provide expression in pulmonary epithelial cells in animal models (K. L. Brigham et al., Am. J. Med. Sci, 298:278-281 (1989); A. B. Canonico et al., Am. J. Respir. Cell. Mol. Biol. 10:24-29 (1994)). Additionally, studies have demonstrated that intramuscular injection of plasmid DNA formulated with 5% PVP (50,000 kDa) increases the level of reporter gene expression in muscle as much as 200-fold over the levels found with injection of DNA in saline alone (R. J. Mumper et al., Pharm. Res. 13:701-709 (1996); R. J. Mumper et al., Proc. Intern. Symp. Cont Rol. Bioac. Mater. 22:325-326 (1995)). Intramuscular administration of plasmid DNA results in gene expression that lasts for many months (J. A. Wolff et al., Hum. Mol. Genet. 1:363-369 (1992); M. Manthorpe et al., Hum. Gene Ther. 4:419-431 (1993); G. Ascadi et al., New Biol. 3:71-81 (1991), D. Gal et al., Lab. Invest. 68:18-25 (1993)).

Additionally, uptake and expression of DNA has also been observed after direct injection of plasmid into the thyroid (M. Sikes et al., Hum. Gene Ther. 5:837-844 (1994)) and synovium (J. Yovandich et al., Hum. Gene Ther. 6:603-610 (1995)). Lower levels of gene expression have been observed after interstitial injection into liver (M. A. Hickman et al., Hum. Gene Ther. 5:1477-1483 (1994)), skin (E. Raz et al., Proc. Natl. Acad. Sci. 91:9519-9523 (1994)), instillation into the airways (K. B. Meyer et al., Gene Therapy 2:450-460 (1995)), application to the endothelium (G. D. Chapman et al., Circulation Res. 71:27-33 (1992); R. Riessen et al., Human Gene Therapy, 4:749-758 (1993)), and after intravenous administration (R. M. Conry et al., Cancer Res. 54:1164-1168 (1994)).

Various devices have been developed for enhancing the availability of DNA to the target cell. A simple approach is to contact the target cell physically with catheters or implantable materials containing DNA (G. D. Chapman et al., Circulation Res. 71:27-33 (1992)). Another approach is to utilize needle-free, jet injection devices which project a column of liquid directly into the target tissue under high pressure. (P. A. Furth et al., Anal Biochem. 20:365-368 (1992); (H. L. Vahlsing et al., J Immunol. Meth. 175:11-22 (1994); (F. D. Ledley et al., Cell Biochem. 18A:226 (1994)).

Another device for gene delivery is the “gene gun” or Biolistic™, a ballistic device that projects DNA-coated micro-particles directly into the nucleus of cells in vivo. Once within the nucleus, the DNA dissolves from the gold or tungsten microparticle and can be expressed by the target cell. This method has been used effectively to transfer genes directly into the skin, liver and muscle (N. S. Yang et al., Proc. Natl. Acad. Sci. 87:9568-9572 (1990); L. Cheng et al., Proc. Natl. Acad. Sci. USA. 90:4455-4459 (1993); R. S. Williams et al., Proc. Natl. Acad. Sci. 88:2726-2730 (1991)).

Another approach to targeted gene delivery is the use of molecular conjugates, which consist of protein or synthetic ligands to which a nucleic acid- or DNA-binding agent has been attached for the specific targeting of nucleic acids to cells (R. J. Cristiano et al., Proc. Natl. Acad. Sci. USA 90:11548-52 (1993); B. A. Bunnell et al., Somat. Call Mol. Genet. 18:559-69 (1992); M. Cotten et al., Proc. Natl. Acad. Sci. USA 89:6094-98 (1992)). Once the DNA is coupled to the molecular conjugate, a protein-DNA complex results. This gene delivery system has been shown to be capable of targeted delivery to many cell types through the use of different ligands (R. J. Cristiano et al., Proc. Natl. Acad. Sci. USA 90:11548-52 (1993)). For example, the vitamin folate has been used as a ligand to promote delivery of plasmid DNA into cells that overexpress the folate receptor (e.g., ovarian carcinoma cells) (S. Gottschalk et al., Gene Ther. 1:185-91 (1994)). The malaria circumsporozoite protein has been used for the liver-specific delivery of genes under conditions in which ASOR receptor expression on hepatocytes is low, such as in cirrhosis, diabetes, and hepatocellular carcinoma (Z. Ding et al., J. Biol. Chem. 270:3667-76 (1995)). The overexpression of receptors for epidermal growth factor (EGF) on cancer cells has allowed for specific uptake of EGF/DNA complexes by lung cancer cells (R. Cristiano et al., Cancer Gene Ther. 3:4-10 (1996)). The presently preferred gene delivery method is lipofection.

It is understood that modifications which do not substantially affect the activity of the various embodiments of this invention are also included within the definition of the invention provided herein. Accordingly, the following examples are intended to illustrate but not limit the present invention.

### EXAMPLE 1

This example describes a representative embodiment of the methods of the invention used to isolated plasmid DNA from E. coli cells.

A 0.5 gram pellet of E. coli cells was suspended in 10 ml of Buffer 1 (50 mM dextrose, 26 mM Tris, 10 mM EDTA, pH 8.0, 50 u/mL β-amylase). To buffer 1 was added 10 mL buffer 2 (0.2 N sodium hydroxide, 0.4% SDS, 0.2% S-485) and the resulting solution incubated for 5 minutes at room temperature. The solution was neutralized by the addition of 10 mL of buffer 3 (3.1 M potassium acetate, pH 5.5). The neutralized bacterial suspension was placed on ice for 10 minutes then centrifuged at 4° C. to yield a pellet and a supernatant.

The supernatant was filtered through a 100 μm cell strainer, and 0.7 volumes of isopropanol (IPA) were added to the strained supernatant. The supernatant was then centrifuged at room temperature to yield a pellet and a supernatant. The pellet was washed with 100% IPA, and then dissolved in 3 mL of buffer 4 (10 mM Tris, pH 8.0, 1u/mL β-amylase). Three mL of buffer 5 (5 M Li CL) were added to the dissolved pallet and the mixture stored at −20° C., and thereafter centrifuged at room temperature to yield a pallet and a supernatant. Six mL of IPA were added to the supernatant which was then centrifuged at room temperature to yield a pellet and a supernatant.

The pellet was dissolved in 1 mL of buffer 6 (10 mM Na₂HPO₄, pH 5.0), 100 μL of buffer 7 (RNase 2 μg/μL in 10 mM Tris, pH 8.0) were added to the dissolved pellet and the mixture incubated at 37° C. for 30 minutes, after which the sample was incubated at 4° C. for 10 minutes. The sample was then loaded onto a Pristine DNA™ column which had been equilibrated in buffer 6 (10 mM Na₂HPO₄, pH 5.0). The following solutions were applied to the column after application of the sample: 4 mL of buffer 6, followed by 5 mL buffer 8 (10 mM Na₂HPO₄, pH 5.0), followed by 10 mL of buffer 9 (0.1 M guanidine-HCL in 10 mM Na₂HPO₄), followed by 5 mL of buffer 8, followed by 2.5 mL of buffer 10 (1 M ethylene diamine/ethylene diamine 2 HCL, pH 8.0

The column was allowed to stand at room temperature for 20 minutes and then a further 5 mL of buffer 2 were added to the column. The resulting column eluate was collected together with the eluate that was produced after application of the previous 12.5 mL of buffer 10 to the column. To the combined eluates was added 12.5 mL IPA, and the resulting solution was then centrifuged at 4° C. to yield a pellet which was washed with 2 mL of cold 70% ethanol, recentrifuged at 4° C. and dried in air for 10 minutes at room temperature. The resulting pellet was dissolved in 0.5 mL buffer 11 (10 mM Tris, 1 mM EDTA, pH 8.0).

Three samples of E. coli plasmid DNA were prepared in accordance with the foregoing isolation procedure (samples C1, C2 and C3 in Table 2). In addition, one sample of E. coli plasmid DNA was prepared in accordance with the foregoing procedure except that the β-amylase was omitted from buffer 4 (sample A in Table 2), and one E. coli plasma DNA sample was prepared in accordance with the foregoing procedure, except that the β-amylase was omitted from buffer 1 (sample B in Table 2).

TABLE 2

| | OD | OD |
|---|---|---|
| | Ratio | Ratio |
| Sample | 260/289 | 269/230 |
| A | 1.59 | 1.26 |
| B | 1.83 | 2.34 |
| C1 | 1.82 | 2.09 |
| C2 | 1.99 | 2.74 |
| C3 | 1.98 | 2.54 |

Agarose gel electrophoresis of the samples set forth in Table 2 revealed that only in sample B was an appreciable amount of nicked plasmid DNA present.

### EXAMPLE 2

This example shows that the use of a hydrophobic surfactant in accordance with the methods of the present invention yields isolated DNA preparations having extremely low levels of bacterial endotoxin.

0.25 grams of pelleted E. coli cells (the cells contained an 8.165 kB plasmid called VR1412) were suspended in the suspension buffer from a Concert Plasmid DNA Purification Kit (Life Technologies, Inc., Bethesda, Md.). In place of the lysis buffer provided in the Concert Plasmid DNA Purification Kit (i.e., 1% SDS in 200 mM NaOH), a series of ten different lysis buffers were evaluated. Each lysis buffer included 1% OT-100 (a hydrophobic surfactant), 0.2M NaOH and from 0.1 to 1% SDS. The lysis buffers were used to lyse the suspended bacterial cells, and the lysed suspension was then neutralized with the neutralization buffer provided in the Concert Plasmid DNA Purification Kit. The resulting plasmid DNA preparation was precipitated by the addition of isopropanol (IPA) followed by centrifugation.

The precipitate was resuspended in 3 mL of a buffer containing 10 mM Tris-HCL, pH 7.0. The following parameters were measured for each of the resuspended samples: A_{260/280}, apparent plasmid DNA concentration (difficult to accurately measure in the crude preparation), endotoxin units per milliliter of sample (EU/mL) as measured using the Limulus Amebocyte Lysate assay (LAL assay, such as the LAL assay kit commercially available from Bio Whittaker, Inc., 8830 Biggs Ford Road, Walkersville, Md. 21793 as catalogue number N204, N488), turbidity of the sample as measured by absorbance at A₆₀₀ and by visual observation of the turbidity. The results are shown in Table 3 wherein the bracketed values in the first two rows are values obtained from repeats of the experiment.

TABLE 3

| | | | Apparent* | | Turbidity of | |
|---|---|---|---|---|---|---|
| | | | Plasmid DNA | EU/mL | Lysis Buffer | Visual |
| Lysis Buffer | A_{260/280 Ratio} | | Conc. | (LAL Assay) | (A₆₀₀) | Observation |
| 1% OT-100, 0.1 SDS | 1.55 | (1.69)(1.58) | 0.612 | 8,169,935 | 0.1248 | Turbid |
| | | | | (531,915)(813,008) | | |
| 1% OT-100, 0.2 SDS | 1.69 | (1.18)(1.64) | 0.812 | 73,892 | 0.2435 | Turbid |
| | | | | (681,199)(6,699,429) | | |
| 1% OT-100, 0.3 SDS | 1.44 | | 1.503 | 119,760 | −0.363 | Turbid |
| 1% OT-100, 0.4 SDS | 1.48 | | 1.196 | 25,084 | 0.01535 | Turbid |
| 1% OT-100, 0.5 SDS | 1.50 | | 0.975 | 5,128,206 | 0.1588 | Turbid |
| 1% OT-100, 0.6 SDS | 1.17 | | 0.111 | 54,054,054 | −0.032 | Turbid |
| 1% OT-100, 0.7 SDS | 1.52 | | 1.497 | 200,401 | −0.058 | Clear (?) |
| 1% OT-100, 0.8 SDS | 1.62 | | 1.286 | 1,555,210 | −0.666 | Clear |
| 1% OT-100, 0.9 SDS | 1.65 | | 1.215 | 411,523 | −0.867 | Clear |
| 1% OT-100, 1.0 SDS | 1.65 | | 1.24 | 201,613 | −0.878 | Clear |

The best result was obtained with 1% OT-100 and 0.4% SDS, that combined a relatively low endotoxin (LAL) level and a high plasmid concentration. Further, 0.4% SDS was the minimum concentration of SDS required to achieve most effective bacterial lysis.

Moreover, mixing the non-hydrophobic surfactant SDS with the hydrophobic surfactant OT-100 yielded a two-phase system, characterized by specific cloud point and viscosity properties not exhibited by SDS alone. The combination of a hydrophobic surfactant and a non-hydrophobic surfactant results in efficient endotoxin removal upon neutralization and plasmid recovery; and relatively low viscosity and reduced non-Newtonian (i.e., viscoelastic) properties when mixing lysate with the neutralization solution, thereby minimizing shear induced degradation of nucleic acids because relatively gentle mixing can be employed. The general trend of going from positive A₆₀₀ values to more and more negative values indicated increasing clarity and hence less phase separation as more SDS was added to 1% OT-100 solution. In general, it is desirable to use the minimum amounts of hydrophobic surfactant and non-hydrophobic surfactant when isolating nucleic acids from cells in order to minimize the amount of these compounds that remain in the isolated nucleic acid preparation.

The results of using various amounts of the hydrophobic surfactant Suffynol-485 (S-485), and a constant amount of the non-hydrophobic surfactant SDS, were investigated as described above using a Concert Plasmid DNA Purification Kit. The results are shown in Table 4.

TABLE 4

| | Plasmid Yield | | | | |
|---|---|---|---|---|---|
| Lysis Buffer | Conc. | Vol. | Yield | | Endotoxin |
| Surfactant | (mg/mL) | (mL) | (mg) | A_{260/280} | (EU/mg) |
| 1% SDS | 1.311 | 0.5 | 0.656 | 1.81 | 1641.5 |
| 0.4% SDS | 1.343 | 0.5 | 0.672 | 1.64 | 2108.0 |
| 0.4% SDS, 0.2% S-485 | 0.968 | 0.5 | 0.484 | 1.74 | 138.0 |
| 0.4% SDS, 0.4% S-485 | 1.264 | 0.5 | 0.632 | 1.69 | 195.7 |
| 0.4% SDS, 0.6% S-485 | 1.942 | 0.5 | 0.971 | 1.62 | 628.0 |
| 0.4% SDS, 0.8% S-485 | 1.608 | 0.5 | 0.804 | 1.86 | 1598.9 |
| 0.4% SDS, 1.0% S-485 | 1.548 | 0.5 | 0.774 | 1.75 | 1621.0 |

A combination of high purity (as assessed by the level of endotoxin) and high yield was obtained with as little as 0.2% Surfynol 485.

### EXAMPLE 3

This example shows that using hydrophobic surfactants in accordance with the methods of the present invention reduces the viscosity, and the extent of the non-Newtonian viscosity properties, of E. coli cell lysates.

A bacterial pellet was suspended in a suspension buffer, to which was added a lysis solution and then a neutralization solution. Viscosity measurements were made at one or more of these stages using a rheometer (Bohlin Instruments, East Brunswick, N.J.). The viscosity of individual suspension, lysis and neutralization solutions were also measured. Viscosity measurements were made as follows: the rheometer was turned on and airbed flow established. The controlling software was set-up to apply a range of shear forces (every third value over about half of the entire range was measured). Samples of approximately 9.5 mL were loaded into the barrel of the rheometer, and the platform/spindle was carefully lowered until it was exactly 0.5 mm from the bottom using the Mitutoyo gauge. The torque bar safety set screws were unlocked and the torque range adjusted to about 99% to ensure that it was not bottomed out. A sample was then measured over a period of about 7.5 minutes. Viscosity was measured at various points whilst increasing the shear rate, and at various points while decreasing the shear rate from an initially high shear rate.

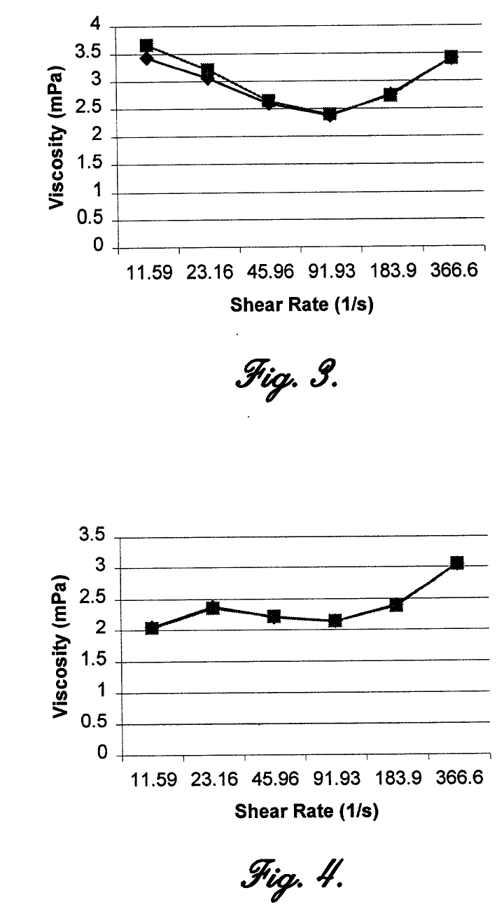

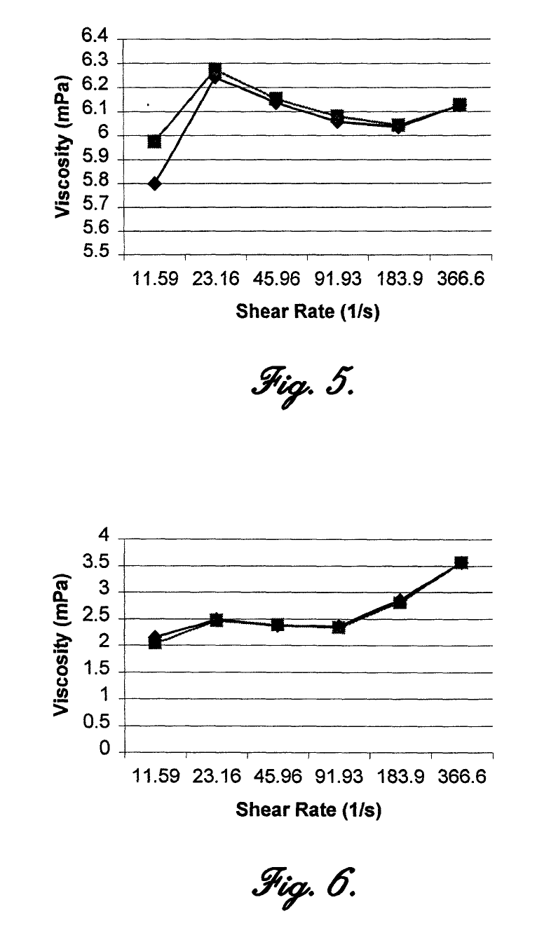

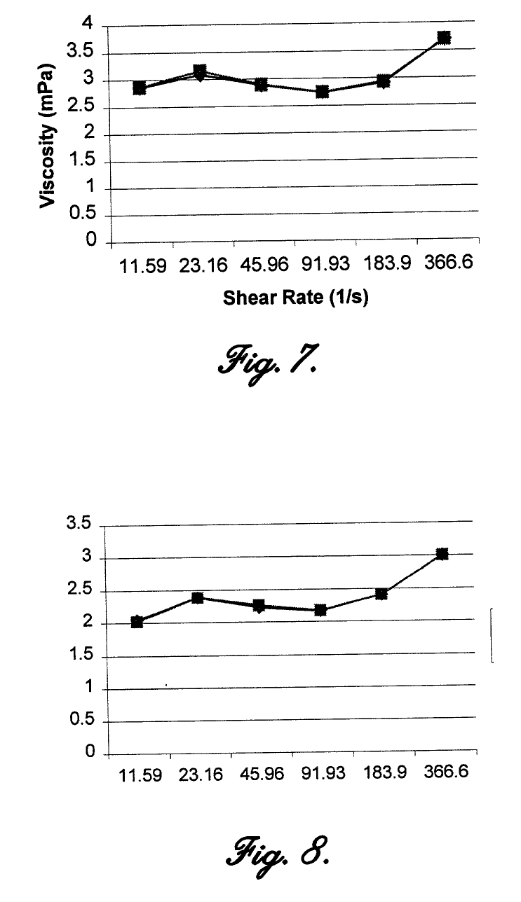

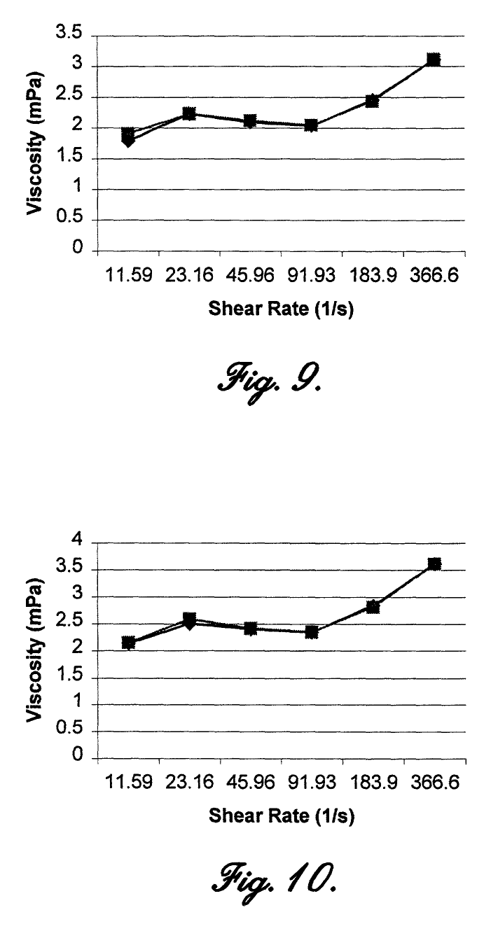

FIGS. 3-10 show plots of viscosity versus shear rate for a variety of suspension, lysis and neutralization solutions having the compositions set forth in the figure legends. In general, none of the suspension, lysis and neutralization solutions tested exhibited non-Newtonian viscosity properties, and their viscosities at a variety of shear rates were within a fairly narrow range of values.

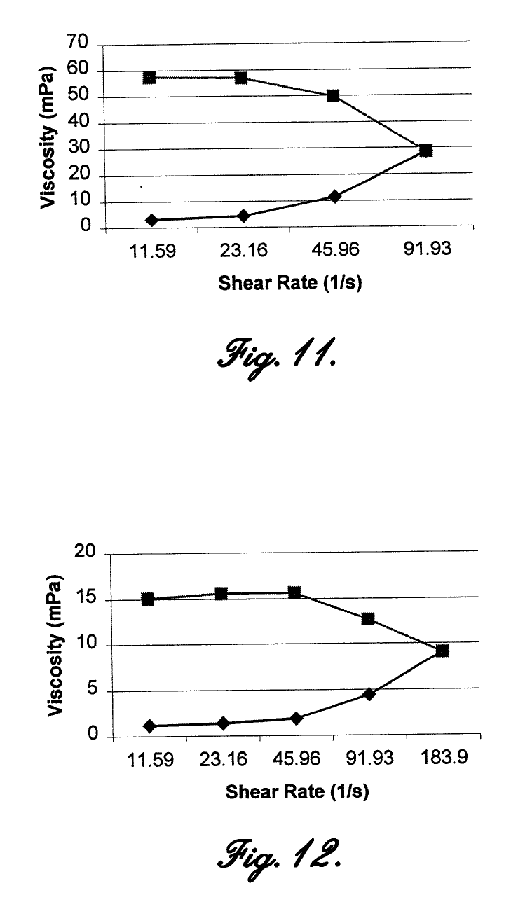

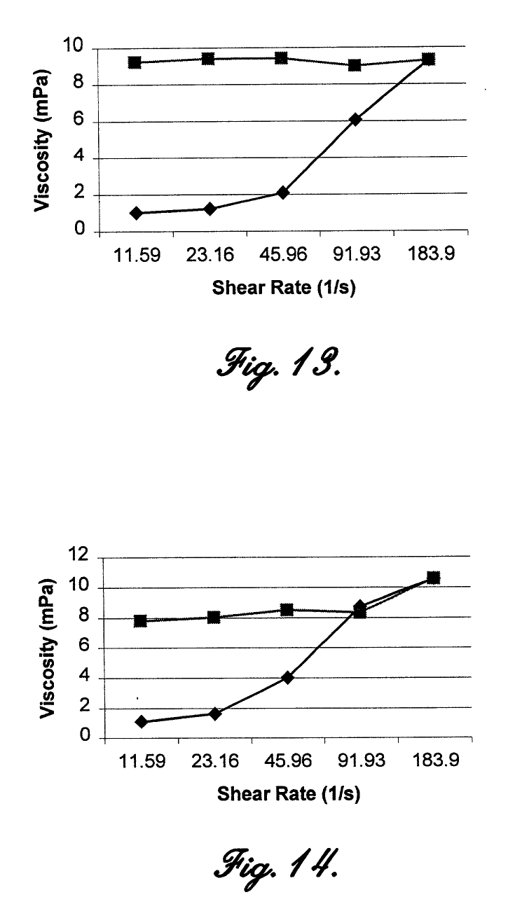

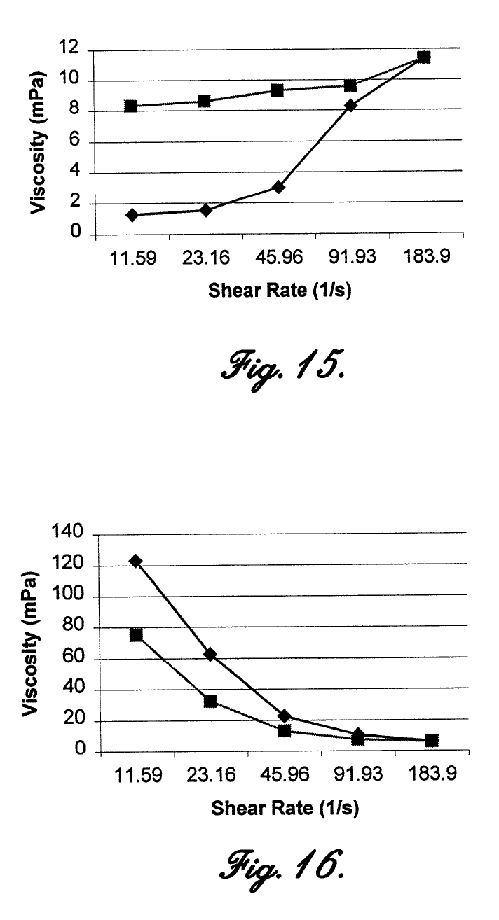

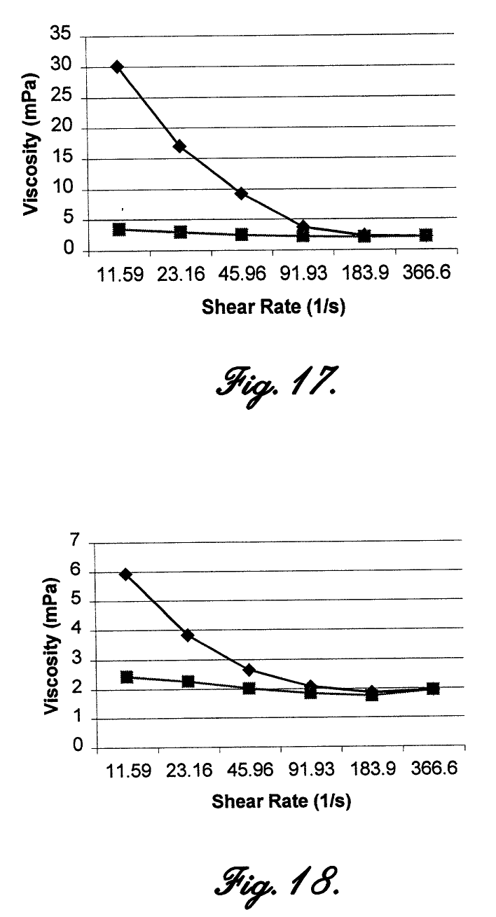

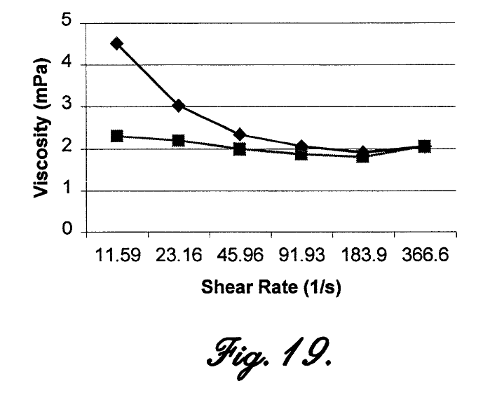

FIGS. 11-15 show plots of viscosity versus shear rate for various combinations of lysis solution plus suspension solution plus E. coli cell extract. As shown in FIG. 11, the viscosity properties of the lysate produced using a conventional lysis solution (1.0% SDS, 0.2 N NaOH), which did not contain a hydrophobic surfactant, was highly non-Newtonian, and was characterized by dramatic shear thickening and shear thinning. In contrast, and as shown in FIGS. 12-15, the lysates produced in accordance with the present invention, using lysis solutions containing a hydrophobic surfactant (e.g., S-485) and, optionally, a non-hydrophobic surfactant (e.g., SDS), exhibited reduced viscosities (and reduced non-Newtonian viscosity behavior) relative to the viscosity properties of the lysate produced using a conventional lysis solution (see FIG. 11). Similarly, as shown in FIG. 16, the magnitude of the viscosity (and the extent of the non-Newtonian viscosity behavior) of the neutralized lysate produced using a conventional lysis solution, that did not include a hydrophobic surfactant, was substantially greater than the magnitude of the viscosities (and the extent of the non-Newtonian viscosity behavior) of neutralized lysates produced in accordance with the methods of the present invention (see FIGS. 17-19).

### EXAMPLE 4

This example shows the effect of different hydrophobic surfactants on the quality of plasmid (VR1412) DNA isolated from 0.5 grams E. coli bacteria in accordance with the method set forth in Example 1 (except that no β-amylase was utilized).

SDS was utilized as the non-hydrophobic surfactant in each experiment at a concentration of 0.4%. The concentration of hydrophobic surfactant was 0.2% in each experiment. The A_{260/280} ratio, the A_{260/230} ratio, the concentration and the yield of isolated plasmid DNA was measured for each experiment. All of the lysis solutions included 0.2 N NaOH. The results are shown in Table 5.

TABLE 5

| | | | Conc | Yield |
|---|---|---|---|---|
| Lysis Buffer Composition | A_{260/280} | A_{260/230} | (mg/mL). | (μg) |
| 0.4% SDS, 0.2% S-485 | 1.69 | 1.63 | 0.61 | 305 |
| 1% SDS | 1.70 | 1.84 | 0.59 | 295 |
| 0.4% SDS, 0.2% S-465 | 1.78 | 2.06 | 0.53 | 265 |
| 0.4% SDS, 0.2% Triton X-100 | 1.70 | 1.74 | 0.61 | 305 |
| 0.4% SDS, 0.2% Tween 20 | 1.73 | 1.96 | 0.56 | 280 |
| 0.4% SDS, 0.2% Tween 80 | 1.83 | 0.57 | 0.43 | 215 |
| 0.4% SDS, 0.2% OT-100 | 1.82 | 1.82 | 0.6 | 300 |

The highest purity DNA was obtained by using a lysis solution including 0.4% SDS and 0.2% S465.

### EXAMPLE 5

This example describes a method for expressing plasmid DNA in mammalian cells in vitro.

SW480 P3 (ATCC #CCL228) human colon carcinoma cells (typically, 1×10⁶ cells) are plated in the wells of a 6-well tissue culture plate. The number of wells plated reflects the number of days post-transfection during which the transfection experiment will proceed. Each well contains 1 ml of complete media from a 30 ml stock solution containing: 26.4 ml RPMI tissue culture medium, 4 mM L-glutamine, 3.0 ml fetal bovine serum, and 10 μg/ml gentamicin. Cells are cultured at 37° C. in a CO₂ incubator with 10% CO₂ for 24 hours after being plated, during which time the cells adhere to the plates.

After the 24 hour pre-incubation step, the transfection step is carried out by removing the RPMI and adding 900 μL OPTI-MEM® (Gibco) medium containing 2 μg of the plasmid DNA, and 8 μg of a mixture of cationic lipid (1,2-dimyristyloxyproply-3-dimethyl-hydroxyethyl ammonium bromide (e.g., “DMRIE/DOPE”) mixed in equimolar proportions with dioleoylphosphatidylethanolamine) to yield a lipid:DNA molar ratio of 0.99:1. It should be noted that typical transient transfection protocols employ 10 μg DNA per 10₆ cells, but the protocol described here uses less DNA in order to reduce toxicity to the cells. The plates are then incubated for 4 hours at 37° C.

After the 4 hour incubation step, 100 μl of heat deactivated fetal bovine serum (to stop transfection), plus 12.0 μl of 50 mg/ml gentamicin are added to each well. At each time point thereafter, all of the cells from one well are trypsinized and counted, then 2×10⁴ cells from each well are lysed and stored in liquid N₂ until used to determine the plasmid expression level.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for isolating nucleic acid molecules from a cell, the method comprising:
(a) contacting a cell with a solution comprising a biopolymer-degrading enzyme, provided that said biopolymer-degrading enzyme is not a nuclease; and
(b) contacting the cell with a solution comprising a hydrophobic surfactant to yield a cell suspension comprising cell, biopolymer-degrading enzyme and hydrophobic surfactant, wherein said hydrophobic surfactant has a critical micelle concentration less than 3.0 mM and the concentration of said hydrophobic surfactant in said cell suspension is at least 0.05% (v/v).

2. The method of claim 1 wherein the solution comprising a biopolymer-degrading enzyme and the solution comprising a hydrophobic surfactant are the same solution.

3. The method of claim 1 wherein the solution comprising a biopolymer-degrading enzyme and the solution comprising a hydrophobic surfactant are different solutions.

4. The method of claim 3 wherein the cell is first contacted with the solution comprising a biopolymer-degrading enzyme, and the cell is next contacted with the solution comprising the hydrophobic surfactant.

5. The method of claim 3 wherein the cell is contacted with the solution comprising a biopolymer-degrading enzyme at the same time that the cell is contacted with the solution comprising the hydrophobic surfactant.

6. The method of claim 1 wherein the biopolymer-degrading enzyme is selected from the group consisting of a carbohydrate-degrading enzyme, a protein-degrading enzyme and a lipid-degrading enzyme.

7. The method of claim 1 wherein the biopolymer-degrading enzyme is a carbohydrate-degrading enzyme.

8. The method of claim 7 wherein the carbohydrate-degrading enzyme is selected from the group consisting of α-amylase, β-amylase, amyloglucosidase, invertase and glycopepsidase F.

9. The method of claim 1 wherein the concentration of said hydrophobic surfactant in said cell suspension is at least 0.1% (v/v).

10. The method of claim 1 wherein the concentration of said hydrophobic surfactant in said cell suspension is at least 0.15% (v/v).

11. The method of claim 1 wherein the concentration of said hydrophobic surfactant in said cell suspension is at least 0.2% (v/v).

12. The method of claim 1 wherein said hydrophobic surfactant has a critical micelle concentration of less than 2.0 mM.

13. The method of claim 1 wherein said hydrophobic surfactant has a critical micelle concentration of less than 1.0 mM.

14. The method of claim 1 wherein said hydrophobic surfactant has a critical micelle concentration of less than 0.5 mM.

15. The method of claim 1 wherein said hydrophobic surfactant has a critical micelle concentration of less than 0.1 mM.

16. The method of claim 1 wherein said hydrophobic surfactant has a hydrophile lipophile balance number of less than 20.

17. The method of claim 1 wherein said hydrophobic surfactant has a hydrophile lipophile balance number of less than 15.

18. The method of claim 1 wherein said hydrophobic surfactant has a solubility of less than 2 g/100 mL in water

19. The method of claim 1 wherein said hydrophobic surfactant has a solubility of less than 1.5 grams/100 ml in water.

20. The method of claim 1 wherein said hydrophobic surfactant has a solubility of less than 1.0 grams/100 ml in water.

21. The method of claim 1 wherein said hydrophobic surfactant is selected from the group consisting of polyoxyethylene sorbitan monolaurate, polyoxyethylene sorbitan monooleate, Triton X-100, Triton X-114, N-tetradecyl-N, N-dimethyl-3-ammonio-1-propanesulfonate, sodium dioctyl sulfosuccinate, surfynol®420, surfynol®440, surfynol®465 and surfynol®485 and TR-70.

22. The method of claim 1 wherein:
(a) said solution comprising a hydrophobic surfactant further comprises a non-hydrophobic surfactant, wherein said non-hydrophobic surfactant has a critical micelle concentration greater than 3.0 mM; and
(b) the concentration of said non-hydrophobic surfactant in said cell suspension is at least 0.4% (v/v).

23. The method of claim 22 wherein the concentration of said non-hydrophobic surfactant in said cell suspension is at least 0.5% (v/v).

24. The method of claim 22 wherein the concentration of said non-hydrophobic surfactant in said cell suspension is at least 0.6% (v/v)

25. The method of claim 22 wherein said non-hydrophobic surfactant has a critical micelle concentration greater than 5.0 mM.

26. The method of claim 22 wherein said non-hydrophobic surfactant has a critical micelle concentration greater than 7.0 mM.

27. The method of claim 22 wherein said non-hydrophobic surfactant has a hydrophile lipophile balance number of greater than 20.

28. The method of claim 22 wherein said non-hydrophobic surfactant has a hydrophile lipophile balance number of greater than 30.

29. The method of claim 22 wherein said non-hydrophobic surfactant has a solubility of greater than 2 grams/100 ml water.

30. The method of claim 22 wherein said non-hydrophobic surfactant is selected from the group consisting of sodium dodecyl sulfate, and CHAPS.

31. The method of claim 22 wherein the concentration of hydrophobic surfactant in said cell suspension is 0.2% (v/v) and the concentration of said non-hydrophobic surfactant in said cell suspension is 0.4% (v/v).

32. A method for isolating nucleic acid molecules from a cell comprising:
(a) suspending a cell in a solution comprising a carbohydrate-degrading enzyme to form a cell suspension;
(b) adding to said cell suspension (1) an amount of at least one hydrophobic surfactant sufficient to yield a hydrophobic surfactant concentration of at least 0.05% (v/v), said hydrophobic surfactant having a critical micelle concentration of less than 3.0 mM, and (2) an amount of an alkaline agent sufficient to increase the pH of said solution to a pH value greater than 10.0; and
(c) adding to said cell suspension prepared in accordance with steps (a) and (b) an amount of a neutralizing agent sufficient to adjust the pH of said solution to within the range of from pH 6.5 to pH 7.5.

33. The method of claim 32 wherein said hydrophobic surfactant has a hydrophile lipophile balance number of less than 20.

34. The method of claim 32 wherein said hydrophobic surfactant has a solubility of less than 2 grams/100 ml in water.

35. The method of claim 32 wherein:
(a) said carbohydrate-degrading enzyme is selected from the group consisting of α-amylase, β-amylase, amyloglucosidase, invertase and glycopepsidase F;
(b) said cell is contacted with said hydrophobic surfactant for a period of from 3 minutes to 12 minutes before adding said neutralizing agent; and
(c) said neutralizing agent is an acidic salt.

36. The method of claim 32 wherein step (b) further comprises adding to the cell suspension of step (a) an amount of a non-hydrophobic surfactant sufficient to yield a non-hydrophobic surfactant concentration of at least 0.4% (v/v), wherein said non-hydrophobic surfactant has a critical micelle concentration greater than 3.0 mM.

37. The method of claim 36 wherein said non-hydrophobic surfactant has a hydrophile lipophile balance number greater than 20.

38. The method of claim 36 wherein said non-hydrophobic surfactant has a solubility greater than 2 grams/100 ml in water.

39. The method of claim 36 wherein:
(a) said hydrophobic surfactant has:
1. a hydrophile lipophile balance number less than 20;
2. a solubility less than 2 grams per 100 ml in water; and said non-hydrophobic surfactant has:
(a) a hydrophile lipophile balance number greater than 20; and
(b) a solubility greater than 2 grams per 100 ml in water.

40. The method of claim 1 wherein said cell is a prokaryotic cell.

41. The method of claim 1 wherein said cell is a eukaryotic cell.

42. The method of claim 1 further comprising the step of isolating nucleic acid having an A₂₆₀/A₂₃₀ ratio of at least 2.0.

43. The method of claim 42 wherein said nucleic acid is DNA.

44. The method of claim 42 wherein said nucleic acid is plasmid DNA.

45. An isolated nucleic acid preparation having an A_{260/230} ratio of at least 2.0, said isolated nucleic acid preparation prepared by any one of the methods of claim 1, claim 22, and claim 32.

46. An isolated DNA preparation comprising at least 80% supercoiled DNA.

47. An isolated DNA preparation of claim 46 comprising at least 90% supercoiled DNA.

48. An isolated DNA preparation of claim 46 comprising at least 95% supercoiled DNA.

49. An isolated DNA preparation comprising at least 80% supercoiled DNA, said DNA preparation being prepared by any one of the methods of claim 1, claim 22, and claim 32.

50. Isolated plasmid DNA that encodes a protein, said isolated plasmid DNA possessing the property of expressing the protein for a period of ten days after introduction into a mammalian cell in vitro, the level of protein expression during the ten day expression period (a) reaching a peak protein expression level and (b) never dropping below 50% of the peak protein expression level after reaching the peak protein expression level.

51. Isolated plasmid DNA of claim 50 wherein the isolated plasmid DNA possesses the property of expressing the protein for a period of fifteen days after introduction into a mammalian cell in vitro, the level of protein expression during the fifteen day expression period (a) reaching a peak protein expression level and (b) never dropping below 50% of the peak protein expression level after reaching the peak protein expression level.

52. Isolated plasmid DNA of claim 50 wherein the isolated plasmid DNA possesses the property of expressing the protein for a period of twenty days after introduction into a mammalian cell in vitro, the level of protein expression during the twenty day expression period (a) reaching a peak protein expression level and (b) never dropping below 50% of the peak protein expression level after reaching the peak protein expression level.

53. Isolated plasmid DNA that encodes a protein, said isolated plasmid DNA:
(a) being isolated from a cell by a method comprising contacting a cell with a solution comprising a biopolymer-degrading enzyme, provided that said biopolymer-degrading enzyme is not a nuclease, and contacting the cell with a solution comprising a hydrophobic surfactant to yield a cell suspension comprising cell, biopolymer-degrading enzyme and hydrophobic surfactant, wherein said hydrophobic surfactant has a critical micelle concentration less than 3.0 mM and the concentration of said hydrophobic surfactant in said cell suspension is at least 0.05% (v/v); and
(b) possessing the property of expressing the protein for a period of time, after introduction into a mammalian cell in vivo, during which time period the level of protein expression (a) reaches a peak protein expression level and (b) never drops below 50% of the peak protein expression level after reaching the peak protein expression level, said time period being at least two times longer than any period of expression of reference plasmid DNA, in the same type of mammalian cell in vivo, during which expression period protein expression does not fall below 50% of the value of the peak protein expression level after reaching the peak protein expression level, said reference plasmid DNA being the same plasmid DNA as the claimed plasmid DNA except that the reference plasmid DNA is prepared by purification twice on a cesium chloride gradient instead of in accordance with the method of (a) herein.
